(19) United States
(12) Patent Application Publication
(10) Pub. No.: US 2017/0348123 A1
(43) Pub. Date: Dec. 7, 2017

(54) BALLOON-MOUNTED STENT DEPLOYMENT APPARATUS AND METHOD OF USE THEREOF

(71) Applicants: GIL MEDICAL CENTER, Incheon (KR); GACHON UNIVERSITY OF INDUSTRY-ACADEMIC COOPERATION FOUNDATION, Seongnam-si (KR)

(72) Inventors: Jae Hee Cho, Incheon (KR); Yoon Jae Kim, Incheon (KR)

(21) Appl. No.: 15/535,291

(22) PCT Filed: Dec. 18, 2015
(86) PCT No.: PCT/KR2015/013943

§ 371 Date: Jun. 12, 2017

(30) Foreign Application Priority Data
Dec. 29, 2014 (KR) 10-2014-0191918

(51) Int. Cl. A61F 2/958; A61B 6/00

(52) U.S. Cl. CPC A61F 2/958; A61B 6/481; A61F 2002/9583

(57) ABSTRACT

The present invention relates to a balloon-mounted stent configured such that a balloon is formed at the front end of a stent, and a metal stent is deployed to a precise stenotic region to widen the stenotic region, and the present invention provides a balloon-mounted stent deployment apparatus comprising an inner tube, a balloon connected to the outside of the inner tube, and a stent located at one side of the balloon and installed outside the inner tube, in which, when the balloon is inflated, the balloon acts as a stopper for the stent. Therefore, during stent deployment procedures, the position of the stent in a stenotic region can be determined more accurately and easily.

### TECHNICAL FIELD

The present invention relates to a balloon-mounted stent deployment apparatus, and more specifically, to a balloon-mounted stent configured such that a balloon is formed at the front end of a stent, and a metal stent is inserted to a precise stenotic region to widen the stenotic region.

### BACKGROUND ART

In general, stent deployment is a technique involving a therapeutic apparatus used in digestive diseases and various vascular diseases. In particular, when a metal stent is inserted to a stenotic region, the stenotic region is widened so that body fluids, blood, and food can flow down smoothly, and this procedure is one of the most important treatment methods. In order to perform such stent deployment, a deployment and separation system, to which a stent is attached, is necessary.

Conventionally, there is a deployment catheter having a stent located at a distal end part, in order to insert a stent to a stenotic region such as a biliary tree. In this case, a pusher catheter is used to deploy the stent from the deployment catheter, and the stent is attached at a front end part of a stent intubation and separation delivery system with a minimal volume. The stent is delivered and separated to a required location by a method of pushing the stent outside the front end part of a deployment catheter by a pusher catheter.

Korean Patent Publication No. 2002-0089342 provides a medical apparatus which is controllable from the proximal region to exert a force on the pusher member and includes a pusher assembly which is mounted in the catheter, in which the distal region of the catheter and apparatus is more flexibly curved on side surfaces as compared to the remaining region, and includes a pusher member for applying a force to a proximal end part of a stent so as to enable smooth delivery of the stent.

Although a stent delivery system of such configuration compensates for the problem of becoming severely twisted between the pusher and the stent during a procedure, thereby allowing smooth delivery, it is difficult to identify the exact stenotic region when the stent deployment procedure is performed clinically, which may result in improper stent deployment.

In fact, although it is necessary to expand the exact stenotic region, cases frequently occur in which it is often difficult due to technical difficulties, etc. to visualize whether the stent is inserted to the stenotic region. In particular, in the cases of esophagus, stomach, duodenum, and bile duct stenosis, stent deployment is performed by endoscopic approach. However, since patients must maintain a posture for endoscopy, it is often difficult to identify the exact stenotic region by radiographic X-ray fluoroscopy alone. Accordingly, a dangerous situation occurs in which the stent is separated from the deployment catheter in a condition in which the operator cannot accurately determine the position of the stent.

Therefore, there is a need for a stent deployment apparatus capable of easily inserting a stent into a stenotic region and separating the stent after determining an accurate position of the stenotic region.

### DISCLOSURE

### Technical Problem

The present invention has been made to solve the above problems.

More specifically, it is an object of the present invention to provide a stent deployment apparatus configured to be able to insert a stent at an accurate position with respect to a stenotic region.

In addition, it is an object of the present invention to provide a stent deployment apparatus configured to more easily determine whether a stent is located at a stenotic region through radiographic X-ray fluoroscopy before a stent is separated from the stent deployment apparatus during a stent insertion procedure.

### Technical Solution

In order to accomplish the above objects, the present invention provides a balloon-mounted stent deployment apparatus, comprising an inner tube 100; a balloon 110 connected to the outside of the inner tube 100; and a stent 200 located at one side of the balloon 110 and installed outside the inner tube 100, in which, when the balloon 110 is inflated, the balloon 110 acts as a stopper for the stent 200.

In addition, it is preferable that the inner tube 100 comprises a passage 111 connected to the balloon 100, and a contrast agent is injected into the balloon 110 through the passage 111.

In addition, it is preferable that the balloon 110 is formed of a transparent flexible material. Further, it is preferable that the balloon 110 comprises balloons 110a and 110b formed at both ends of the stent 200.

In addition, the inner tube 100 comprises a passage 111 connected to the balloons 110a and 110b, and it is preferable that a contrast agent is injected to the balloons 110a and 110b through the passage 111.

In addition, it is preferable that the passage 111 is provided with an opening/closing means inside, and the opening/closing means controls the access of the contrast agent into and from each of the balloons 110a and 110b.

In addition, it is preferable that a coating agent is coated on the surface of the balloon 110.

In addition, it is preferable that at least one of the balloons (110a and 110b) is coated with a coating agent on the surface.

In addition, the present invention provides a method for using a balloon-mounted stent deployment apparatus, comprising: (a) inserting an inner tube 100 mounted with a stent 200 at a predetermined position; (b) inflating a balloon 110 located on one side of the stent 200 and mounted on the inner tube 100; (c) determining the predetermined position and the position of the balloon 110 on an image processing apparatus; and (d) expanding the stent 200 by deflating the balloon 110 and removing the inner tube 100 from the predetermined position, when the balloon is positioned at a desired position with respect to the predetermined position.

In addition, it is preferable that step (b) above comprises (b1) inflating at least one of balloons 110a and 110b.

### Advantageous Effects

According to the balloon-mounted stent deployment apparatus according to the present invention as described above, the stent deployment apparatus is mounted with a balloon injected with a contrast agent or gas, so that the position of a stent in a stenotic region can be more accurately and easily determined during a stent insertion procedure.

In addition, it is possible to obtain clearer radiographic X-ray fluoroscopic images by using materials such as a contrast agent, and at the same time perform the procedure in which the contrast agent does not remain in the human body after a stent insertion procedure.

### BRIEF DESCRIPTIONS OF THE DRAWINGS

FIG. 1 is a schematic diagram of a balloon-mounted stent deployment apparatus according to an embodiment of the present invention.

FIG. 2 is a diagram illustrating a balloon-mounted stent deployment apparatus according to an embodiment of the present invention including a partial enlarged view in which part of the front end is cut and shown by a diagram.

FIG. 3 is a schematic diagram for explaining an operational method of a balloon-mounted stent deployment apparatus according to an embodiment of the present invention.

FIG. 4 a schematic diagram for explaining an operational method of a balloon-mounted stent deployment apparatus according to another embodiment of the present invention.

### DETAILED DESCRIPTION OF THE INVENTION

The balloon-mounted stent deployment apparatus of the present invention will be described as an example. The configurational elements constituting the present invention can be used integrally or used by being individually separated, as required. Further, some components may be omitted and used depending on the usage form.

Hereinafter, the present invention will be described in detail with reference to the drawings.

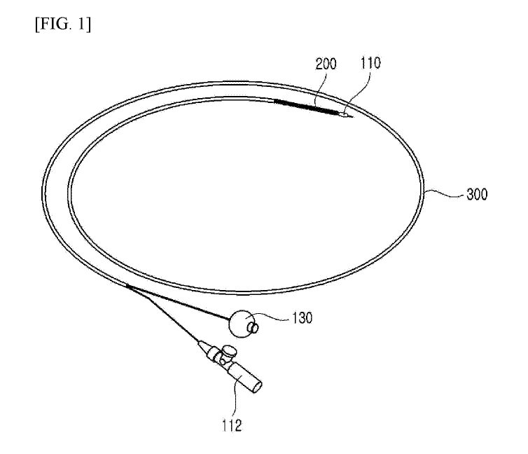

A preferred exemplary embodiment of the balloon-mounted stent deployment apparatus according to the present invention will be described with reference to FIGS. 1 to 4. In this process, the thicknesses of the lines and sizes of the components, etc. shown in the drawings may be exaggerated for clarity and convenience of explanation. Further, the terms to be described below are terms defined in consideration of the functions of the present invention, and these may vary depending on the user, intention of the operator, or custom. Therefore, definitions of these terms should be described based on the content throughout the present specification.

Hereinafter, an embodiment of the balloon-mounted stent deployment apparatus according to the present invention will be described with reference to the accompanying drawings.

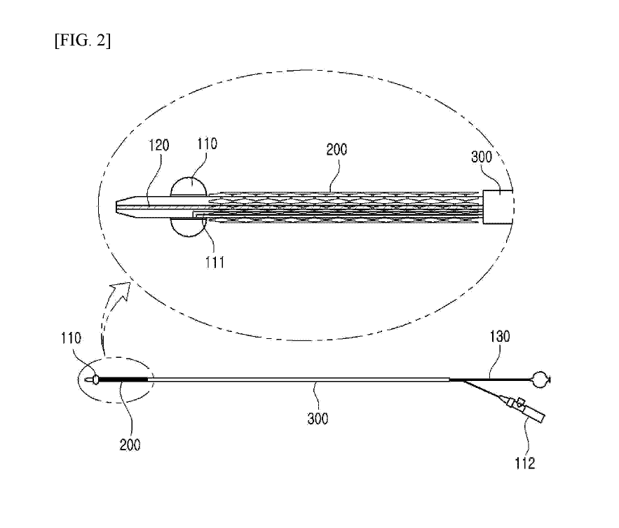

First, the configuration of an endoscope apparatus will be described with reference to FIGS. 1 and 2. The balloon-mounted stent according to an embodiment of the present invention comprises an inner tube 100, a balloon 110 connected to the outside of the inner tube 100, and a stent 200 located at one side of the balloon 110 and installed outside the inner tube 100.

The inner tube 100 comprises a passage 111 connected to the balloon 110, and a contrast agent or gas may be injected into the balloon 110 through a hub 112 located at the rear end part of the deployment apparatus and the passage 111. When a contrast agent or gas is injected into the balloon 110, the balloon is inflated, and the position of the inflated balloon 110 may be more clearly displayed through an image processing apparatus. Further, if the balloon 110 acts as a stopper at a predetermined position such as a stenotic region while the balloon 110 is inflated and the inflated balloon 110 is caught by a part of the stenotic region and cannot easily be pulled out, the operator can recognize whether the deployment in the stenotic region is successful without reliance on images. In this case, the image processing apparatus may include an apparatus capable of radiographic X-ray fluoroscopy. In this case, the balloon 110 is positioned at one side of the stent 200 at the front end part of the stent deployment apparatus so that the position of the stent 200 can be easily determined.

The passage 111 is preferably formed in the inner tube 100 and connected to the hub 112 located at the rear end part of the deployment apparatus. The hub 112 preferably comprises an inlet for a gas or contrast agent, which is connected to the passage 111, and it is also preferable to comprise a regulating valve capable of regulating the injected gas or contrast agent.

It is preferable that another passage is formed in the inner tube 100 as shown in FIG. 2 so that a guide wire 120 can be inserted inside. A part of the front end part of the deployment apparatus shown in FIG. 2 is partially cut to explain the guide wire 120. Since the guide wire 120 is formed in the inner tube 100, it is easy to control the deployment apparatus to be positioned at a predetermined position.

The balloon 110 is preferably formed of a transparent flexible material, and although the transparent flexible material is not limited by any one kind, it is preferably formed of a material harmless to the human body. Since the balloon 110 is formed of a transparent flexible material, the position of the balloon 110 on radiographic X-ray fluoroscopic images can be more easily determined when a contrast agent is injected into the balloon 110.

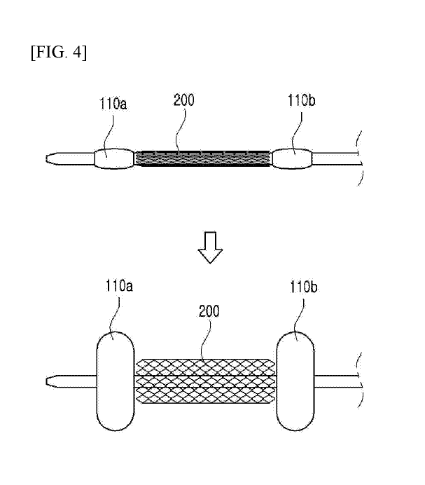

In addition, the balloon 110 may be formed at both ends of the stent 200, and in particular, on the basis of an inserted position, since a balloon 110b which is located behind the stent 200 (see FIG. 4) is transparent, an endoscope camera is not obscured by the balloon 110b when inserted in a stenotic region, so that the front end part of the stent deployment apparatus can be seen.

When balloons 110a and 110b are formed at both ends of the stent 200, the passage 111 is preferably formed to connect to both balloons 110a and 110b. It is preferable that the contrast agent or gas is injected into the balloons 110a and 110b through the passage 111. It is preferable that the passage 111 is provided with an opening/closing means inside, and is configured to control the access of a contrast agent or gas into each of the balloons 110a and 110b. The opening/closing means may be various valves and is not limited to any one kind.

In another embodiment, the balloon may be coated with a contrast agent on the surface and used. When the balloons 110a and 110b are formed on both sides of the stent 200, a contrast agent may be coated on the surface of any one or both of the balloons 110a and 110b. Accordingly, the amount of the contrast agent used can be reduced, and radiographic X-ray fluoroscopic images can be displayed more clearly. The contrast agent may be coated on the inside of the balloons 110a and 110b when the balloons 110a and 110b are formed of a transparent material.

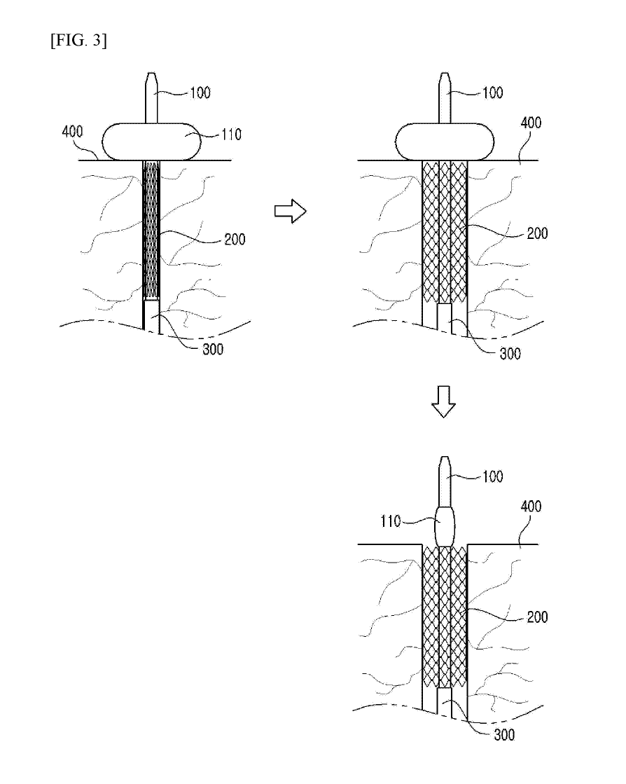

Next, a method for using a balloon-mounted stent deployment apparatus will be described with reference to FIGS. 3 and 4. As a method for using the balloon-mounted stent deployment apparatus, the inner tube 100 mounted with the stent 200 is first deployed into a stenotic region. In this case, it is preferable that the stent 200 is in close contact with the outer surface of the inner tube 100.

The approximate position of the stenotic region can be determined through a camera of an endoscope apparatus, and when the stent 200 reaches the stenotic region, a contrast agent or gas may be injected into the balloon 110 to determine whether the stent 200 is inserted or not. Accordingly, the balloon 110 can be inflated and displayed more easily in radiographic X-ray fluoroscopic images.

When the position of the balloon 110 is recognized in images, it is possible to determine whether the stent 200 is separated. The position of the stent 200 close to the balloon 110 can be determined according to the position of the balloon 110, and when the stent 200 is positioned at a predetermined position, the contrast agent or gas that has been injected into the balloon 110 passes through the passage 111 and further passes through the hub 112 which is connected thereto and is exhausted, thereby deflating the balloon 110.

When the balloon 110 is deflated, a handle 130 located at the rear end of the inner tube 100 may be used to separate the stent 200 from the stent deployment apparatus. By pulling the handle 130 toward the back of the stent in the direction opposite to the insertion direction of the stent 200, the position of the stent 200 is maintained because the stent 200 is held by a part of an outer tube 300, and only the inner tube 100 is moved so that the stent 200 can be separated from the inner tube 100. However, various modifications are possible in the manner in which the stent 200 is separated from the inner tube 100.

When the inner tube 100 is removed from the stenotic region, the stent can be expanded to widen the stenotic region. In this case, if the balloons 110a and 110b are formed on both sides of the stent according to another embodiment, any one of the balloons 110a and 110b may be inflated according to the shape or position of the stenotic region as needed, and may be selectively inflated.

While the present invention has been explained above with reference to exemplary embodiments thereof, it will be understood by those skilled in the art that various changes and modifications may be made in the present invention without departing from the spirit and scope of the present invention as defined within the scope of the following claims.

### DESCRIPTION OF REFERENCE NUMERALS

100: Inner tube

110, 110a, 110b: Balloon

111: Passage

112: Hub

120: Guide wire

130: Handle

200: Stent

300: Outer tube

1. A balloon-mounted stent deployment apparatus, comprising:
an inner tube;
a balloon connected to the outside of the inner tube; and
a stent located at one side of the balloon and installed outside the inner tube,
wherein when the balloon is inflated, the balloon acts as a stopper for the stent.

2. The balloon-mounted stent deployment apparatus of claim 1, wherein the inner tube comprises a passage connected to the balloon, and a contrast agent is injected into the balloon through the passage.

3. The balloon-mounted stent deployment apparatus of claim 1, wherein the balloon is formed of a transparent flexible material.

4. The balloon-mounted stent deployment apparatus of claim 3, wherein the balloon comprises balloons formed at both ends of the stent.

5. The balloon-mounted stent deployment apparatus of claim 4, wherein the inner tube comprises a passage connected to the balloons, and a contrast agent is injected into the balloons through the passage.

6. The balloon-mounted stent deployment apparatus of claim 5, wherein the passage is provided with an opening/closing means inside, and the opening/closing means controls the access of the contrast agent into and from each of the balloons.

7. The balloon-mounted stent deployment apparatus of claim 1, wherein a contrast agent is coated on the surface of the balloon.

8. The balloon-mounted stent deployment apparatus of claim 4, wherein at least one of the balloons is coated with a contrast agent on the surface.

9. A method for using a balloon-mounted stent deployment apparatus, comprising:
(a) inserting an inner tube mounted with a stent at a predetermined position;
(b) inflating a balloon located on one side of the stent and mounted on the inner tube;
(c) determining the predetermined position and the position of the balloon on an image processing apparatus; and
(d) expanding the stent by deflating the balloon and removing the inner tube from the predetermined position, when the balloon is positioned at a desired position with respect to the predetermined position.

10. The method for using a balloon-mounted stent deployment apparatus of claim 9, wherein step (b) comprises:
(b1) inflating at least one of balloons.
